(19) United States
(12) Patent Application Publication
(10) Pub. No.: US 2026/0242876 A1
(43) Pub. Date: Aug. 20, 2026

(54) METHOD FOR DETERMINING THE SUSCEPTIBILITY OF A STRAIN OF MYCOBACTERIUM TUBERCULOSIS TO PYRAZINAMIDE

(71) Applicant: BIOMÉRIEUX, Marcy L’etoile (FR)

(72) Inventors: Pierre MAHE, Lans en Vercors (FR); Maud TOURNOUD, Sassenage (FR); Philippine BARLAS, La Buisse (FR); Magali JAILLARD DANCETTE, Lyon (FR)

(73) Assignee: BIOMÉRIEUX, Marcy L’etoile (FR)

(21) Appl. No.: 19/160,726

(22) PCT Filed: Mar. 7, 2024
(86) PCT No.: PCT/EP2024/056055

§ 371 Date: Aug. 28, 2025

(30) Foreign Application Priority Data
Mar. 10, 2023 (EP) 23161246.6

(51) Int. Cl. C12Q 1/689; C12Q 1/6809; G16B 20/20; G16B 30/00

(52) U.S. Cl. CPC C12Q 1/689; C12Q 1/6809; G16B 20/20; G16B 30/00; C12Q 2600/156

(57) ABSTRACT

A method for determining the susceptibility of a strain of Mycobacterium tuberculosis to pyrazinamide, the method includes the following steps: (i) determining the presence in the genome of this strain of at least one mutation in the rpoB gene between positions 761112 and 761182; and (ii) determining the sensitive or resistant nature of this strain depending on the presence or absence of the at least one mutation. The invention also relates to a kit including means for detecting at least one sequence chosen from among the sequences SEQ ID NO: 1 to 3 and to the use of this same kit for determining the susceptibility of a strain of Mycobacterium tuberculosis to pyrazinamide.

### FIELD OF THE INVENTION

The present invention relates to the technical field of molecular biology applied to bacterial genomics, and in particular to the field of determining antibiotic resistance from the analysis of their genome. The invention relates in particular to a method for determining the resistance of a strain of the species Mycobacterium tuberculosis to a particular antibiotic, namely pyrazinamide.

### TECHNOLOGICAL BACKGROUND

Tuberculosis (TB) is an infectious disease of the human respiratory tract that affects nearly one third of the world's population and is a global health problem.

Mycobacterium tuberculosis is the bacterium responsible for tuberculosis and, although vaccines are available, their effect decreases over time and infected patients are generally treated with antibiotics such as isoniazid, rifampicin or pyrazinamide to control the disease.

It belongs to the genus Mycobacteria (Mycobacterium spp.), like the leprosy bacillus (Mycobacterium leprae or Hansen's bacillus), or what are referred to as atypical mycobacteria, and was discovered by Robert Koch in 1882 and its genome was sequenced in 1998.

In recent years, the development of multi-drug resistant Mycobacterium tuberculosis strains, which are highly resistant and fully resistant to drugs such as antibiotics, has been reported.

Multi-drug antibiotic resistance involves resistance of the microbial strain to any first-line antituberculosis drug, including isoniazid and/or rifampicin, but may also involve resistance to any second-line drug, for instance pyrazinamide.

Inadequate or delayed antibiotic therapy has a negative effect because it promotes the selection of spontaneous mutations in favor of resistant strains by selection pressure, thereby exacerbating the problem of antibiotic resistance. Therefore, the development of rapid and accurate detection of the resistance profile of Mycobacterium tuberculosis is of global public health interest.

The susceptibility of a bacterial strain to an antibiotic, i.e. its sensitivity or resistance in the context of a treatment based on the antibiotic administered to a human or an animal, cannot be directly observed by a human being. Indeed, direct observation of the strain, even using microscopes, does not make it possible to determine its behavior toward the antibiotic.

In vitro diagnosis in a bacterial context consists, by nature, in making this phenotypic nature observable and therefore ultimately exploitable for a clinician. During the 20th century, in vitro diagnostic technologies essentially combined culture-based sample preparation techniques, in particular to make the bacterial strains present in the samples visible and manipulable, and techniques for optical measurement of the behavior of the strains in the presence of an antibiotic.

For example, a conventional microbiology laboratory workflow involves first spreading a sample, taken from a patient suspected of having a bacterial infection, on a culture medium in order to cause bacterial colonies that are visible to a human operator or an automated system to appear after incubation. Secondly, when the colonies are large enough, a technician or an automated system samples a colony, mixes it with an antibiotic at different concentrations and introduces the mixtures into a device that measures the optical density of each mixture and deduces the susceptibility to the antibiotic therefrom. Since optical density indicates bacterial proliferation, it therefore unambiguously characterizes the sensitivity or resistance of the bacterium: if the density increases, this means that it is proliferating despite the presence of the antibiotic, and thus that it is resistant to said antibiotic at the concentration of the antibiotic under consideration.

For example, document U.S. Pat. No. 7,335,485 describes a method for determining the susceptibility of a microorganism to an antibiotic, wherein the microorganism is cultured in the presence of the antibiotic to be tested.

The combination of sample preparation technologies and optical density-based measurement technologies has significant limitations in the face of rapid global evolution in the prokaryotic kingdom, namely the acquisition of multi-drug resistance to antibiotics, which is predicted to be responsible for more deaths than cancer by 2050. Depending on the chosen culture medium, certain strains will grow and others will not, and as such these technologies do not make it possible to characterize the antibiotic susceptibility of all bacterial species. In addition, these techniques are extremely slow since they are based on bacterial culturing that takes a long time. Thus, obtaining an antibiogram of a bacterium takes at least 30 hours from the time the sample is taken. This delay does not enable effective treatment of patients, who are systematically given a broad-spectrum antibiotic cocktail as a first-line treatment. In addition to the consequences for the patient, this inappropriate and massive administration of antibiotics reinforces the selection pressure of multi-drug-resistant bacteria and thus contributes to their expansion.

Thus, at present, conventional in vitro diagnostic technologies are considered to be increasingly unsuitable for treating patients and are, to a certain extent, one of the reasons for the emergence of multi-drug resistance.

More recently, sensitive technologies such as mass spectrometry have been applied to determine antibiotic resistance, but this still requires culturing the microorganism to be tested in the presence of the antibiotic to be tested. In addition, in all these techniques, each microorganism to be tested must be tested against individual antibiotics or combinations of antibiotics, requiring extensive, time-consuming and tedious tests.

The maturation of molecular biology technologies, in particular bacterial DNA and/or RNA characterization technologies, such as polymerase chain reaction (PCR), DNA chips or sequencing, is bringing about a paradigm shift in the analysis of antibiotic resistance in laboratories.

Firstly, they are more agnostic in respect of bacterial species. For example, metagenomic technology makes it possible to process bacterial DNA in a biological sample irrespective of the bacterial species present. Secondly, they aim to provide a result in a few hours, with some, such as PCR, even providing a result in less than 20 minutes. On the other hand, molecular techniques for characterizing antibiotic susceptibility are based on genomic signatures (absence/presence of genes, genetic mutations, predictive models, etc.) characterizing said susceptibility.

Non-limitingly, in the context of a microbiological workflow for treating a patient suspected of having a bacterial infection, there are two technologies for characterizing bacterial DNA, namely PCR technology and whole-genome sequencing (WGS) technology. Both workflows begin by taking a biological sample from the patient, followed by applying PCR or WGS technology, each yielding a result of genomic signatures that characterize susceptibility to one or more antibiotics, on the basis of which result an antibiotic treatment is chosen and administered to the patient by a clinician. Conventionally, according to the methods known to those skilled in the art, each of the molecular technologies requires the preparation of the sample taken before the application of the PCR itself, for example, a “nested” PCR implemented by a FilmArray® platform from BioFire®, or before the application of the sequencing, for example a sequencing of the SBS type (Sequencing by Synthesis) implemented by a MiSeq platform from Illumina.

Document WO2018/065830 describes, for example, a quantitative real-time PCR (qPCR) method for determining the resistance profile of Mycobacterium tuberculosis to antibiotics.

Document CN101580879 describes a gene chip and a method for detecting common genetic mutations of Mycobacterium tuberculosis which are responsible for resistance to antibiotics such as isoniazid, rifampicin, streptomycin, ethambutol or pyrazinamide.

Document IN201941006113 describes a method for testing antibiotic resistance of a Mycobacterium tuberculosis strain from a clinical sample containing said strain, by implementing a step of identifying single nucleotide variants (SNVs) from genes associated with the antibiotic resistance of said strain.

More generally, in both bacteria and humans, it is known that antibiotic resistance can be associated with gene polymorphism. Focusing more particularly on genomic signatures, the first approaches consisted in identifying previously identified antibiotic resistance markers in the bacterial genome, referred to as “direct association” approaches. While these approaches are effective when the genetic mechanisms leading to resistance are well known and simple, they can suffer from significant drawbacks: incomplete knowledge of the mechanisms of resistance in many species and in many antibiotics, reflected for example in incomplete databases, the difficulty of taking into account differences in the predictive powers of markers and the multifactorial aspect of antibiotic susceptibility (e.g. epistasis, combination of multiple mutations, etc.), etc.

In the face of these challenges, the genetic determinism of antibiotic susceptibility is more effectively understood by new approaches based on advanced computer technologies, and in particular by supervised machine learning technologies, the learning and application architecture of which can be summarized as follows:
A. for a set of training bacterial strains:
   A.1 each strain is sequenced and phenotypically characterized (e.g. measurement of its minimum inhibitory concentration and/or measurement of its susceptibility—resistance, intermediate or sensitive—to one or more antibiotics).
   A.2. a computer model for predicting the susceptibility to the antibiotic is trained on the basis of the genomes and phenotypic data.
B. for a new strain for which the susceptibility to an antibiotic from step (A.1) is sought:
B.1. the strain is sequenced;
B.2. the predictive computer model is applied to its digital genome so as to determine its susceptibility.

Such learning and predictive models are described, for example, in documents WO2021180768 and WO2021180771 in the name of the Applicant.

There is therefore a need to develop new genomic signatures in order to reinforce the arsenal available for the identification of susceptibility to antibiotics, and in particular to pyrazinamide. Indeed, signatures are obtained by learning models, the limits of which lie in the size and diversity of the genomic bases used to train the model. Therefore, it may be advantageous to combine the simultaneous use of multiple signatures in order to determine susceptibility to an antibiotic with an even greater level of accuracy. In this context, it is relevant to identify new genomic signatures which have a sufficient level of performance, namely in particular a specificity of at least 90%, in determining the susceptibility of a strain of the species Mycobacterium tuberculosis to pyrazinamide.

### SUMMARY OF THE INVENTION

After extensive research on the genome of the Mycobacterium tuberculosis species, it is to the inventors' credit to have identified a new genomic signature that makes it possible to determine the susceptibility of a strain of said species to pyrazinamide. In other words, the determination of susceptibility according to the present invention makes it possible to identify whether a strain of the Mycobacterium tuberculosis species is sensitive or resistant to pyrazinamide with a specificity of more than 90%. Thus, the present invention makes it possible to combat antibiotic resistance, in particular by avoiding initiating pyrazinamide treatment when the strain is resistant.

Moreover, it is known that the presence of mutations in the pncA gene is correlated with the resistance of strains of the species Mycobacterium tuberculosis to pyrazinamide. Conversely, and entirely surprisingly, the sensitivity or resistance of a Mycobacterium tuberculosis strain to pyrazinamide is determined from the identification of the presence or absence of mutations in genes that are not constitutively correlated with resistance to said antibiotic, such as the rpoB gene or else the gyrA, embB or katG genes.

A first subject of the invention thus relates to a method for determining the susceptibility of a strain of the species Mycobacterium tuberculosis to pyrazinamide, said method comprising the steps of determining the presence, in the genome of said strain, of at least one mutation in the rpoB gene between positions 761112 and 761182, and determining the sensitivity or resistance of said strain on the basis of the identified mutation(s).

Preferably, the method also comprises the steps of determining the presence or absence of at least one mutation in the fabG1 gene between positions 1673413 and 1673454, optionally also in the in the gyrA gene between positions 7552 and 7582, optionally also in the Rv1042c gene and the promoter region thereof, between positions 1165444 and 1165528, optionally also in the Rv1149 gene and the promoter region thereof, between positions 1277873 and 1277957, optionally also in the embB gene between positions 4247581 and 4247622, optionally also in the pncA gene between positions 2288853 and 2289239, and finally optionally also in the katG gene between positions 2155164 and 2155205.

Another subject of the invention relates to a kit comprising means for detecting and/or amplifying at least one sequence selected from the sequences SEQ ID NOs: 1 to 3, and optionally at least one other sequence selected from the sequences SEQ ID NOs: 4 to 12, in the genome of a strain of the Mycobacterium tuberculosis species, preferably in the rpoB gene of said strain, and also to the use of said kit for determining the susceptibility of the strain to pyrazinamide.

Since the method according to the invention can be implemented by a computer, another subject finally relates to a data processing apparatus, comprising:
(a) means for implementing the method according to the invention, in particular means for determining the presence of mutations in the rpoB gene of the Mycobacterium tuberculosis strain between positions 761112 and 761182, and/or means making it possible to compare the sequence of said gene with the reference genome Mycobacterium tuberculosis H37Rv (reference NC_000962.3), and/or means for identifying the presence of the sequences SEQ ID NOs: 1 to 3 in the genome of the Mycobacterium tuberculosis strain, and/or means for providing output data regarding the presence or absence of said sequences when said means are implemented or controlled by a computer, or
(b) a processor suitable for, or configured for, executing the computer-implemented method as claimed in claim 16, in particular a processor suitable for, or configured for, executing the steps of said method.

### DETAILED DESCRIPTION OF THE INVENTION

Pyrazinamide is a nicotinamide derivative and an antituberculosis drug which makes it possible to obtain, in vivo and at the indicated dosages, a bactericidal action on intracellular tuberculosis bacilli (the bacilli are thus in an acidic medium, which is a prerequisite for the action of pyrazinamide). Pyrazinamide is therefore used in tuberculosis management. The species Mycobacterium bovis and atypical mycobacteria are naturally resistant to pyrazinamide. Conversely, the species Mycobacterium tuberculosis and a highly similar species, Mycobacterium africanum, are commonly sensitive to pyrazinamide.

As mentioned above, inadequate or delayed antibiotic therapy has a negative effect by promoting the selection of spontaneous mutations in favor of resistant strains by selection pressure, thereby exacerbating the problem of antibiotic resistance.

Therefore, when managing a patient with tuberculosis, it is essential to be able to ensure that the Mycobacterium tuberculosis strain with which said patient is infected is not resistant to pyrazinamide.

Thus, a first subject of the invention relates to a method for determining the susceptibility of a strain of the species Mycobacterium tuberculosis to pyrazinamide, said method comprising a step of determining the presence, in the genome of said strain, of at least one mutation in the rpoB gene between positions 761112 and 761182, and a step of determining the sensitivity or resistance of said strain on the basis of the presence or absence of mutations in said gene.

Entirely surprisingly, as explained above, the present method makes it possible to determine susceptibility to pyrazinamide based on the identification of mutations in genes that are not constitutively associated with pyrazinamide resistance but rather with resistance to other antibiotics. Indeed, the identification of mutations in the rpoB gene is normally associated with resistance to rifamycin and derivatives thereof, such as rifampicin.

Unless explicitly specified, the technical and scientific terms used in the present description have the same meaning as that which is commonly understood by those skilled in the art in the field of the present description.

The term “determining susceptibility” refers to the determination of the sensitivity or resistance of the strain to pyrazinamide.

For the remainder of the description, the term “strain(s)” refers to strains of the species Mycobacterium tuberculosis, unless the context makes it possible to clearly identify that reference is being made to another species.

Determining the Presence of at Least One Mutation in the rpoB Gene

The term “mutation” refers to a variation in the sequence with respect to a reference sequence. Such a reference sequence may be a sequence determined in a predominant wild-type organism or a reference organism such as a defined and known bacterial strain, for example. A mutation is, for example, a deletion of one or more nucleotides, an insertion of one or more nucleotides, or a substitution of one or more nucleotides, a duplication of a nucleotide or of a sequence of several nucleotides, a translocation of a nucleotide or of a sequence of several nucleotides, and, in particular, a single nucleotide polymorphism (SNP).

The genome of Mycobacterium tuberculosis was fully sequenced in 1998. This species has a circular chromosome of 4 411 529 base pairs (GC %=65.6) for 3924 genes.

In the context of the present invention, the presence of mutations in one or more genes of the strain is determined with respect to the wild-type reference genome of Mycobacterium tuberculosis, namely Mycobacterium tuberculosis H37Rv. This wild-type reference genome is in particular indexed in the NCBI database under the reference NC_000962.3. Thus, all the genomic positions according to the present description are given with respect to said genome NC_000962.3.

The comparison can be carried out according to methods known to those skilled in the art for comparing two genomes with each other, or more precisely, particular genomic positions between two genomes, namely in particular by bioinformatics.

Thus, according to the present invention, the determination of the presence, in the genome of the strain, of at least one mutation in the rpoB gene between positions 761112 and 761182 with respect to the wild-type genome Mycobacterium tuberculosis H37Rv, makes it possible to identify the sensitivity or resistance of said strain to pyrazinamide.

For the purposes of the present invention, the identification or determination of the sensitivity or resistance of a Mycobacterium tuberculosis strain is understood as the prediction, with a certain associated degree of error, of said sensitivity or resistance.

The presence of at least one mutation in one or more genes of a strain of Mycobacterium tuberculosis can be determined using methods known to those skilled in the art. For example, it is possible to implement a hybridization, amplification or sequencing method.

According to a particular embodiment, the presence of mutations is determined using a hybridization technique, preferably with hybridization microchips or by techniques such as NanoString® nCounter®, by an amplification technique, preferably by PCR or qPCR, or by a sequencing technique, preferably by high throughput sequencing or synthesis sequencing.

According to a preferred embodiment, the method comprises, before the step of determining the presence of mutations in the genes of the Mycobacterium tuberculosis strain, a prior step of characterizing the DNA of said strain. This step is carried out according to methods known to those skilled in the art, preferably by PCR or sequencing (for example WGS).

The method may also comprise a prior step of obtaining a biological sample from a subject, said sample being liable to contain at least one strain of the Mycobacterium tuberculosis species.

According to a particular embodiment, the step of determining the presence of at least one mutation in the rpoB gene comprises determining the presence of at least one mutation between positions 761151 and 761182, and/or between positions 761112 and 761142, and/or between positions 761138 and 761173.

According to a preferred variant of this embodiment, the step of determining the presence of at least one mutation in the rpoB gene of the strain comprises determining the presence, in this gene, of at least one sequence selected from the sequences SEQ ID NO: 1, SEQ ID NO: 2 and SEQ ID NO: 3, preferably SEQ ID NO: 2, and more preferably still, the sequences SEQ ID NOs 1 to 3. According to the method of the invention, the sensitivity or resistance of the strain is determined on the basis of the presence or absence of mutation(s) between the positions described above, and in particular on the basis of the presence or absence of one or more sequences selected from the sequences SEQ ID NOs: 1 to 3. Table 1 in the exemplary embodiments summarizes the various conclusions regarding the sensitivity or resistance of said strain on the basis of the presence or absence of said sequences.

### Determining the Presence of at Least One Mutation in One or More Additional Genes

The performance of the method according to the present invention can be improved by combining the determination of the presence of mutation(s) in one or more additional genes of the Mycobacterium tuberculosis strain.

Thus, according to a particular embodiment, the method also comprises a step of determining the presence, in the genome of the strain, of at least one mutation in the fabG1 gene between positions 1673413 and 1673454, and preferably between positions 1673415 and 1673454 and/or between positions 1673413 and 1673444.

According to a preferred variant of this embodiment, the determination of the presence of at least one mutation in the fabG1 gene between said genomic positions advantageously comprises determining the presence of the sequence SEQ ID NO: 4 and/or SEQ ID NO: 5, preferably both.

Advantageously, the method according to the invention thus comprises a step of determining, in the genome of a Mycobacterium tuberculosis strain, the presence of at least one mutation in the rpoB and fabG1 genes, said step comprising determining the presence of the sequences SEQ ID NO: 1, SEQ ID NO: 2 and SEQ ID NO: 3 in the rpoB gene and of the sequences SEQ ID NO: 4 and SEQ ID NO: 5 in the fabG1 gene, and also a step of determining the sensitivity or resistance on the basis of the presence or absence of said sequences. Table 2 in the exemplary embodiments summarizes the various conclusions regarding the sensitivity or resistance of said strain on the basis of the presence or absence of said sequences.

According to another particular embodiment, the method according to the present invention also comprises a step of determining the presence, in the genome of the strain, of at least one mutation in the gyrA gene between positions 7552 and 7582. According to a preferred variant of this embodiment, the determination of the presence of at least one mutation in the gyrA gene between said genomic positions advantageously comprises determining the presence of the sequence SEQ ID NO: 6.

Thus, entirely advantageously, the method according to the invention comprises a step of determining, in the genome of a Mycobacterium tuberculosis strain, the presence of at least one mutation in the rpoB, fabG1 and gyrA genes, said step comprising determining the presence of the sequences SEQ ID NOs: 1 to 6 in said respective genes, and a step of determining the sensitivity or resistance on the basis of the presence or absence of said sequences. Table 3 in the exemplary embodiments summarizes the various conclusions regarding the sensitivity or resistance of said strain on the basis of the presence or absence of said sequences.

According to another embodiment, the method according to the invention also comprises a step of determining the presence, in the genome of the strain, of at least one mutation in the Rv1042c gene and the promoter region thereof, between positions 1165444 and 1165528, and/or at least one mutation in the Rv1149 gene and the promoter region thereof, between positions 1277873 and 1277957.

Preferably, according to this embodiment, the method comprises a step of determining the presence of at least one mutation in the Rv1042c gene and the promoter region thereof, between positions 1165444 and 1165492 and/or between positions 1165497 and 1165528, and determining at least one mutation in the Rv1149 gene and the promoter region thereof, between positions 1277873 and 1277904 and/or between positions 1277909 and 1277957.

According to a first variant of this embodiment, the method comprises a step of determining the presence of at least one mutation in the Rv1042c gene between positions 1165444 and 1165492 and at least one mutation in the Rv1149 gene between positions 1277909 and 1277957. According to this variant, the determination of the presence of at least one mutation in said gene advantageously comprises determining the presence of the sequence SEQ ID NO: 7.

According to a second variant of this embodiment, the method comprises a step of determining the presence of at least one mutation in the Rv1042c gene and the promoter region thereof, between positions 1165497 and 1165528, and at least one mutation in the Rv1149 gene and the promoter region thereof, between positions 1277873 and 1277904. According to this variant, the determination of the presence of at least one mutation in said gene advantageously comprises determining the presence of the sequence SEQ ID NO: 8.

According to a third preferred variant of this embodiment, the determination of the presence of at least one mutation in the Rv1042c and/or Rv1149 gene and the respective promoter regions thereof, between said genomic positions, advantageously comprises determining the presence of the sequence SEQ ID NO: 7 and/or of the sequence SEQ ID NO: 8, preferably both.

Thus, according to another preferred embodiment, the method according to the invention comprises a step of determining, in the genome of a Mycobacterium tuberculosis strain, the presence of at least one mutation in the rpoB, fabG1, gyrA, Rv1042c and Rv1149 genes, said step comprising determining the presence of the sequences SEQ ID NO: 1 to SEQ ID NO: 8 in said respective genes, and a step of determining the sensitivity or resistance on the basis of the presence or absence of said sequences. Table 4 in the exemplary embodiments summarizes the various conclusions regarding the sensitivity or resistance of said strain on the basis of the presence or absence of said sequences.

According to another embodiment, the method according to the present invention also comprises a step of determining the presence, in the genome of the strain, of at least one mutation in the embB gene between positions 4247581 and 4247622. Preferably, according to this embodiment, the determination of the presence of at least one mutation in the embB gene between said genomic positions advantageously comprises determining the presence of the sequence SEQ ID NO: 9.

Thus, according to another preferred embodiment, the method according to the invention comprises a step of determining, in the genome of a Mycobacterium tuberculosis strain, the presence of at least one mutation in the rpoB, fabG1, gyrA, Rv1042c, Rv1149 and embB genes, said step comprising determining the presence of the sequences SEQ ID NO: 1 to SEQ ID NO: 9 in said respective genes, and a step of determining the sensitivity or resistance on the basis of the presence or absence of said sequences. Table 5 in the exemplary embodiments summarizes the various conclusions regarding the sensitivity or resistance of said strain on the basis of the presence or absence of said sequences.

According to another embodiment, the method according to the present invention also comprises a step of determining the presence, in the genome of the strain, of at least one mutation in the pncA gene between positions 2288853 and 2289239, and preferably between positions 2288853 and 2288885 and/or between positions 2289209 and 2289239.

According to a preferred variant of this embodiment, the determination of the presence of at least one mutation in the pncA gene between said genomic positions advantageously comprises determining the presence of the sequence SEQ ID NO: 10 and/or of the sequence SEQ ID NO: 11.

Thus, according to another preferred embodiment, the method according to the invention comprises a step of determining, in the genome of a Mycobacterium tuberculosis strain, the presence of at least one mutation in the rpoB, fabG1, gyrA, Rv1042c, Rv1149, embB and pncA genes, said step comprising determining the presence of the sequences SEQ ID NO: 1 to SEQ ID NO: 11 in said respective genes, and a step of determining the sensitivity or resistance on the basis of the presence or absence of said sequences. Table 6 in the exemplary embodiments summarizes the various conclusions regarding the sensitivity or resistance of said strain on the basis of the presence or absence of said sequences.

According to another embodiment, the method according to the present invention also comprises a step of determining the presence, in the genome of the strain, of at least one mutation in the katG gene between positions 2155164 and 2155205.

According to a preferred variant of this embodiment, the determination of the presence of at least one mutation in the katG gene between said genomic positions advantageously comprises determining the presence of the sequence SEQ ID NO: 12.

According to another preferred embodiment, the method for determining the susceptibility of the strain to pyrazinamide of a strain of the species Mycobacterium tuberculosis comprises the following steps:
determining the presence or absence of at least one mutation in the rpoB gene between positions 761112 and 761182,
determining the presence or absence of at least one mutation in the fabG1 gene between positions 1673413 and 1673454,
determining the presence or absence of at least one mutation in the gyrA gene between positions 7552 and 7582,
determining the presence or absence of at least one mutation in the Rv1042c gene between positions 1165444 and 1165492 and/or in the genomic region between positions 1165497 and 1165528, and at least one mutation in the Rv1149 gene between positions 1277909 and 1277957, and/or in the genomic region between positions 1277873 and 1277904,
determining the presence or absence of at least one mutation in the embB gene between positions 4247581 and 4247622,
determining the presence or absence of at least one mutation in the pncA gene between positions 2288853 and 2289239,
determining the presence or absence of at least one mutation in the katG gene between positions 2155164 and 2155205, and
determining the sensitivity or resistance of said strain on the basis of the presence or absence of said mutations.

According to another preferred embodiment, the method according to the invention comprises a step (a) of determining, in the genome of a Mycobacterium tuberculosis strain, the presence of at least one mutation in the rpoB, fabG1, gyrA, Rv1042c, Rv1149, embB, pncA and katG genes, said step comprising determining the presence of the sequences SEQ ID NOs 1, 4, 6, 7, 9, 10 and 12, and preferably SEQ ID NOs: 1 to 12, in said respective genes, and a step (b) of determining the sensitivity or resistance on the basis of the presence or absence of said sequences. Table 7 in the exemplary embodiments summarizes the various conclusions regarding the sensitivity or resistance of said strain on the basis of the presence or absence of said sequences.

According to a particular embodiment, the method may comprise a step of obtaining the genome of said Mycobacterium tuberculosis strain prior to determining the presence or absence of at least one mutation in the rpoB gene, and optionally in the additional genes.

For the purposes of the present description, the term “subject” denotes a human being, and the subject is preferably a patient.

Thus, the method according to the invention may be an in vitro or ex vivo method for determining the susceptibility of a strain of the species Mycobacterium tuberculosis to pyrazinamide, from a biological sample of a subject liable to contain said strain, said method comprising the following steps:
determining the presence, in the genome of said strain, of at least one mutation in the rpoBgene between positions 761112 and 761182, and
determining the sensitivity or resistance of said strain on the basis of the presence or absence of said mutation.

Of course, the specific or preferred embodiments described above according to the invention apply to the in vitro or ex vivo methods which are also a subject of the invention. Thus, it is possible to determine at least one mutation in one of the additional genes as described above.

“Biological sample” refers here to any sample from a subject, which may be of different natures, such as blood or derivatives thereof, or sputum, in which it is possible to find traces of the strain.

According to a particular embodiment, the biological sample is a sputum sample, a blood sample or a blood-derived sample, which can be chosen in particular from whole blood (such as is collected venously, i.e. containing white and red cells, platelets and plasma), plasma and serum.

### Kit for Implementing the Method According to the Invention

Kits can be prepared in order to implement the method according to any of the preceding embodiments. In particular, another subject of the invention relates to a kit enabling the detection and/or amplification of at least one sequence selected from the sequences SEQ ID NO: 1-3. The kit can advantageously include operating instructions.

The kit thus comprises means making it possible to detect and/or amplify and/or quantify said sequences, for example in a biological sample suspected of containing a strain of the species Mycobacterium tuberculosis. These means may thus be specific primers and/or probes for said sequences.

According to a preferred embodiment, the kit comprises means for detecting the sequences SEQ ID NO: 1, SEQ ID NO: 2 and SEQ ID NO: 3. Advantageously, the kit contains all the means necessary for the implementation of the detection of the sequences SEQ ID NOs: 1 to 3 by amplification, preferably by PCR, and in particular by qPCR.

According to a particular embodiment, the kit also comprises means, preferably primers and/or probes, making it possible to detect at least one of the sequences SEQ ID NOs: 4 to 12 in a strain of the species Mycobacterium tuberculosis. Preferably, the kit contains primers and/or probes for detecting all of the sequences SEQ ID Nos 4 to 12.

All of the specific or preferred embodiments described above in association with the methods according to the invention also apply to the kit which is a subject of the invention.

“Primer” or “amplification primer” means a nucleotide fragment which may consist of 5 to 100 nucleotides, preferably 10 to 20 nucleotides, and having hybridization specificity with a target nucleotide sequence, i.e. one of the sequences SEQ ID NOs 1 to 12, under conditions determined for the initiation of enzymatic polymerization, for example in a reaction for the enzymatic amplification of the target nucleotide sequence. Generally, use is made of “primer pairs” consisting of two primers. When it is desired to amplify several different biomarkers (e.g. from different genes), several different pairs of primers are preferably used, each preferentially having the ability to hybridize specifically with a different biomarker.

Those skilled in the art are thus able to use the sequences SEQ ID NOs: 1 to 12 to determine the primers and probes needed for amplification.

“Probe” or “hybridization probe” means a nucleotide fragment typically consisting of 5 to 100 nucleotides, preferably 10 to 90 nucleotides, even more preferably 15 to 35 nucleotides, having hybridization specificity under conditions determined for forming a hybridization complex with a target nucleotide sequence. The probe also includes a reporter (such as a fluorophore, an enzyme or any other detection system) which will enable the detection of the target nucleotide sequence. In the present invention, the target nucleotide sequences are the sequences SEQ ID NOs 1 to 3, and preferably the sequences SEQ ID NOs 1 to 12. Thus, several different probes are preferably used, each preferentially having the ability to hybridize specifically with one of the target sequences.

“Hybridization” means the process during which, under suitable conditions, two nucleotide fragments, for instance a hybridization probe and a target nucleotide fragment, having sufficiently complementary sequences, are able to form a double strand with stable and specific hydrogen bonds. A nucleotide fragment that is “able to hybridize” with a polynucleotide is a fragment which can hybridize with said polynucleotide under hybridization conditions, which can be determined in each case in a known manner. The hybridization conditions are determined by the stringency, i.e. the strictness of the operating conditions. Hybridization is proportionately more specific the higher the stringency levels at which it is performed. Stringency is in particular defined on the basis of the base composition of a probe/target duplex, and also by the degree of mismatch between two nucleic acids. Stringency can also be based on the reaction parameters, such as the concentration and type of ionic species present in the hybridization solution, the nature and the concentration of denaturing agents, and/or the hybridization temperature. The stringency of the conditions under which a hybridization reaction must be performed will chiefly depend on the hybridization probes used. All this information is well known and the suitable conditions can be determined by those skilled in the art.

In general, depending on the length of the hybridization probes used, the temperature for the hybridization reaction is between approximately 20 and 70° C., in particular between 35 and 65° C. in a saline solution at a concentration of approximately 0.5 to 1 M. A step of detecting the hybridization reaction is subsequently carried out.

Probes or primers that may be used in the methods of the invention may typically be short nucleic acid molecules, e.g. DNA oligonucleotides which are 10 nucleotides or more in length and which can be linked to the complementary target nucleic acid molecule by nucleic acid hybridization so as to form a hybrid between the primer or probe and the target nucleic acid strand.

The probe or primers may be unlabeled or labeled so that binding thereof to a target sequence can be detected (for example with a FRET-like donor or acceptor label).

A primer may be extended along the target nucleic acid molecule by a polymerase enzyme.

Accordingly, primers can be used to amplify the target nucleic acid molecule, such as one of the sequences SEQ ID NOS: 1 to 12, and/or the variant sequences thereof.

The specificity of a probe or a primer increases with its length. Thus, for example, a probe or primer that comprises 30 consecutive nucleotides will bind to a target sequence with greater specificity than a corresponding primer of only 15 nucleotides. Thus, to achieve greater specificity, it is possible to select probes and primers that comprise at least 15, 20, 25, 30, 35, 40, 45, 50, 55, 60, 65, 70 or more consecutive nucleotides.

In particular examples, a primer may be at least 15 nucleotides in length, for instance at least 15 contiguous nucleotides that are complementary to a target nucleic acid molecule. Particular primer lengths that can be used to perform the methods of the present disclosure include primers having at least 15, at least 16, at least 17, at least 18, at least 19, at least 20, at least 21, at least 22, at least 23, at least 24, at least 25, at least 26,

at least 27, at least 28, at least 29, at least 30, at least 31, at least 32, at least 33, at least 34, at least 35, at least 36, at least 37, at least 38, at least 39, at least 40, at least 45, at least 50, at least 55, at least 60, at least 65, at least 70, or more contiguous nucleotides that are complementary to the target nucleic acid molecule to be amplified, such as a primer of 15-70 nucleotides, 15-60 nucleotides, 15-50 nucleotides, or 15-30 nucleotides.

A “sense” (or forward) primer is a primer located 5′ with respect to a reference point on a nucleic acid sequence. An “antisense” (or reverse) primer is a primer located 3′ with respect to a reference point on a nucleic acid sequence. In general, at least one sense primer and one antisense primer (the “primer pair”) are included in an amplification reaction.

Nucleic acid probes and primers, or primer pairs, can be readily prepared on the basis of the nucleic acid sequence of any of the sequences SEQ ID NOs 1-12. The PCR primer pairs can be derived from said sequences using computer programs provided for this purpose, such as Primer 3 (v. 0.4.0, Whitehead Institute for Biomedical Research, Steve Rozen and Helen Skaletsky).

According to a particular embodiment, the sequences SEQ ID NOs 1-12, or the amplified or transformed products thereof (cDNA for example) are detected by specific hybridization of nucleic acid probes.

These probes can also be immobilized on a solid surface (such as nitrocellulose, glass, quartz, fused silica slide) such as in an array, microarray or DNA chip. Those skilled in the art are able to recognize that the precise sequence of particular probes and primers can be modified to a certain extent based on the target sequence in order to produce probes that are “substantially identical” or “substantially complementary” to a target sequence, while retaining the ability to bind specifically (i.e. hybridize specifically) to the same targets from which they are derived.

In the context of the present description, the terms “able to hybridize to” and “specifically binds to”, which are used interchangeably, refer to a polynucleotide sequence that forms Watson-Crick bonds with a complementary sequence. Depending on the length of the polynucleotides, the length of the complementary region and the stringency of the conditions, those skilled in the art understand that the percentage of complementarity does not necessarily have to be 100% for hybridization or specific binding to occur. For example, a primer or probe is at least 60%, 70%, 80%, 90%, 95%, 99% or 100% complementary along the length of the complementary region.

Another subject of the invention relates to the use of the kit as defined above for determining the susceptibility of a Mycobacterium tuberculosis strain to pyrazinamide.

Another subject of the invention relates to the use of the sequences SEQ ID NOs 1 to 3, and preferably also sequences SEQ ID NOs 4 to 12, to determine the susceptibility of a Mycobacterium tuberculosis strain to pyrazinamide.

Of course, the specific or preferred embodiments described in association with the methods according to the invention apply to the uses which are also a subject of the invention.

### Computer Implementation

The method as described according to any embodiment detailed above can of course be computer-implemented. In this case, it can be partially or totally executed by computer means.

To this end, the invention also provides a data processing apparatus, comprising:
(a) means for applying a computer-implemented method according to the invention, in particular means for determining the presence of mutations in the rpoB gene of the Mycobacterium tuberculosis strain and/or means making it possible to compare the sequences of said genes with the reference genome Mycobacterium tuberculosis H37Rv (reference NC_000962.3), and/or means for identifying the presence of the sequences SEQ ID NOs: 1 to 3 in the genome of the Mycobacterium tuberculosis strain, and/or means for providing output data regarding the presence or absence of said sequences when said means are implemented or controlled by a computer
(b) a processor suitable for, or configured for, executing a computer-implemented method according to the invention, in particular a processor suitable for, or configured for, executing the steps of a computer-implemented method according to the invention.

Advantageously, the processing apparatus can also comprise means for determining the presence of mutations in the fabG1, gyrA, Rv1042c, Rv1149, embB, pncA and katG genes at the positions as defined above, and/or means for identifying the presence or absence of the sequences SEQ ID NOs: 4 to 12 in said genes to which they relate.

According to a particular embodiment, such a data processing apparatus comprises:
(a) an input interface for receiving the genome of a Mycobacterium tuberculosis strain for which it is sought to determine the susceptibility to pyrazinamide,
(b) a memory for storing at least instructions for a computer program which, when the program is executed by a computer or a processor, leads to executing the determination of the presence of mutations in the rpoB gene at the positions described above, and optionally in one or more additional genes as described above,
c) a processor that accesses the memory to read the abovementioned instructions and execute a computer-implemented method according to the invention
(d) an output interface for providing the output values, in particular the output values corresponding to a conclusion regarding the sensitivity or resistance of the strain to pyrazinamide.

### Other Subjects of the Description

Another subject of the present description relates to a method for treating a subject infected with a strain of the species Mycobacterium tuberculosis, comprising a step of determining the susceptibility of said strain to pyrazinamide according to any one of the methods as described above, and a step of treating said subject with pyrazinamide once a conclusion has been drawn regarding the sensitivity of said strain.

In particular, processing can be initiated as soon as a conclusion has been drawn regarding the sensitivity.

Another subject of the present description relates to a method comprising the following steps:
obtaining a biological sample from a subject infected with a strain of the species Mycobacterium tuberculosis,
bringing said biological sample into contact with detection means or specific reagents making it possible to detect the presence of mutations in the rpoB of said strain between positions 761112 and 761182, and
determining the presence of the sequences SEQ ID NOs: 1 to 3.

The reagents specific to the expression products are selected from amplification primers and hybridization probes, and are as defined above, and make it possible in particular to determine the presence of the sequences SEQ ID NOs: 1 to 3.

Preferably, the biological sample is also brought into contact with means for determining the presence of mutations in the fabG1, gyrA, Rv1042c, Rv1149, embB, pncA and katG genes at the positions as defined above, and makes it possible in particular to identify the presence or absence of the sequences SEQ ID NOs: 4 to 12 in said genes to which they relate.

The present invention is illustrated, non-limitingly, by the examples below.

### EXAMPLES

### Example 1: Determining the Susceptibility of a Mycobacterium tuberculosis Strain to Pyrazinamide

### 1. Obtaining Models

A set of 3606 genomes of Mycobacterium tuberculosis having phenotypes that are resistant (R) or sensitive (S) to pyrazinamide was used. Of these genomes, 3038 were used to train supervised machine learning models (training set) and 568 were used to evaluate the performance of these models (validation set).

Of the 568 genomes in the validation set, 133 are resistant to pyrazinamide and 435 are sensitive to pyrazinamide. The tables below present, for each model, the presence of each wild-type sequence of the model in these validation genomes. The sequences are given the name of the gene to which they belong, and are ordered by decreasing value of the coefficient in the model (given as an absolute value, in parentheses).

The tables below demonstrate different haplotypes defined as combinations of mutated or non-mutated sequences observed. The haplotypes are ordered by decreasing prevalence (percentage of observations among the 568 genomes).

For each haplotype, the prediction made by the model, and also the number of resistant and sensitive genomes in which this haplotype was observed, are reported. It is thus possible to identify the number of good predictions and the number of errors made by the model in the validation set.

These errors are described in terms of “very major error” (VME) corresponding to a resistant genome predicted to be sensitive by the model (i.e., a false negative) and “major error” (ME) corresponding to a sensitive genome predicted to be resistant by the model (i.e., a false positive).

Legends for tables 1 to 6: NoRS=number of resistant strains; NoSS=number of sensitive strains; Prev=prevalence; Pred=prediction; 1: presence of the sequence; 0: absence of the sequence.

### 2. Application of the Models for Determining Susceptibility to Pyrazinamide

2.1 rpoB Model

Table 1 below presents the susceptibility results by the detection of the presence of mutations in the rpoB gene of the strain by demonstrating the presence or absence of the sequences SEQ ID NOs: 1 to 3.

TABLE 1

| | | | | | | SEQ ID NO: | | |
|---|---|---|---|---|---|---|---|---|
| | | | | | | 2 | 1 | 3 |
| | | | | | | rpoB | rpoB | rpoB |
| NoRS | NoSS | Prev | Pred | ME | VME | (5.3) | (4.33) | (0.94) |
| 7 | 391 | 70.07 | S | 0 | 7 | 1 | 1 | 1 |
| 98 | 23 | 21.3 | R | 23 | 0 | 0 | 1 | 0 |
| 19 | 4 | 4.05 | R | 4 | 0 | 1 | 0 | 1 |
| 3 | 13 | 2.82 | S | 0 | 3 | 1 | 0 | 0 |
| 5 | 3 | 1.41 | S | 0 | 5 | 1 | 1 | 0 |
| 1 | 0 | 0.18 | R | 0 | 0 | 0 | 0 | 0 |

### Interpretation of Results

In this model, the predominant haplotype is the one in which the presence of the sequences SEQ ID NOs: 1 to 3 is observed. This haplotype is observed in 398 genomes (391+7), i.e. 70.07% of the 568 validation genomes. Therefore, determining the presence of the sequences SEQ ID NOs: 1 to 3 leads to the correct determination (or prediction) of the “sensitivity” for 391 genomes, but with 7 VME.

Conversely, the absence of said sequences SEQ ID NOs: 1 to 3 represents the minority haplotype and is observed in a single genome, i.e. 0.18% of the validation genomes. Therefore, determining the absence of the sequences SEQ ID NOs: 1 to 3 leads to the correct determination (or prediction) of the “resistance”, without any ME or VME.

### Performance of the Model

Overall, this model makes it possible to predict, on the basis of the presence or absence of the sequences SEQ ID NOs: 1 to 3, the sensitivity or resistance of a Mycobacterium tuberculosis strain with a specificity of 88.72%, a sensitivity of 93.56% and an error rate of 7.57% (ME and VME).

2.2 rpoB-fabG1 Model

Table 2 below presents the susceptibility results by the detection of the presence of mutations in the rpoB and fabG1 genes of the strain by demonstrating the presence or absence of the sequences SEQ ID NOs: 1 to 5.

TABLE 2

| | | | | | | SEQ ID NO: | | | | |
|---|---|---|---|---|---|---|---|---|---|---|
| | | | | | | 2 | 1 | 4 | 5 | 3 |
| | | | | | | rpoB | rpoB | fabG1 | fabG1 | rpoB |
| NoRS | NoSS | Prev | Pred | ME | VME | (4.99) | (4.1) | (1.02) | (1.02) | (0.79) |
| 6 | 359 | 64.26 | S | 0 | 6 | 1 | 1 | 1 | 1 | 1 |
| 44 | 19 | 11.09 | R | 19 | 0 | 0 | 1 | 1 | 1 | 0 |
| 54 | 4 | 10.21 | R | 4 | 0 | 0 | 1 | 0 | 0 | 0 |
| 1 | 32 | 5.81 | S | 0 | 1 | 1 | 1 | 0 | 0 | 1 |
| 1 | 13 | 2.46 | S | 0 | 1 | 1 | 0 | 1 | 1 | 0 |
| 8 | 4 | 2.11 | R | 4 | 0 | 1 | 0 | 1 | 1 | 1 |
| 11 | 0 | 1.94 | R | 0 | 0 | 1 | 0 | 0 | 0 | 1 |
| 2 | 3 | 0.88 | S | 0 | 2 | 1 | 1 | 1 | 1 | 0 |
| 3 | 0 | 0.53 | S | 0 | 3 | 1 | 1 | 0 | 0 | 0 |
| 2 | 0 | 0.35 | R | 0 | 0 | 1 | 0 | 0 | 0 | 0 |
| 1 | 0 | 0.18 | R | 0 | 0 | 0 | 0 | 1 | 1 | 0 |
| 0 | 1 | 0.18 | R | 1 | 0 | 0 | 1 | 1 | 1 | 1 |

### Interpretation of Results

In this model, the predominant haplotype is the one in which the presence of the sequences SEQ ID NOs: 1 to 5 is observed. This haplotype is observed in 365 genomes (359+6), i.e. 64.26% of the 568 validation genomes. Therefore, determining the presence of the sequences SEQ ID NOs: 1 to 5 leads to the correct determination (or prediction) of the “sensitivity” for 359 genomes, but with 6 VME.

### Performance of the Model

Overall, this model makes it possible to predict, on the basis of the presence or absence of the sequences SEQ ID NOs: 1 to 5, the sensitivity or resistance of a Mycobacterium tuberculosis strain with a specificity of 90.23%, a sensitivity of 93.56% and an error rate of 7.21% (ME and VME).

2.3 rpoB-fabG1-gyrA Model

Table 3 below presents the susceptibility results by the detection of the presence of mutations in the rpoB, fabG1 and gyrA genes of the strain by demonstrating the presence or absence of the sequences SEQ ID NOs: 1 to 6.

TABLE 3

| | | | | | | SEQ ID NO: | | | | | |
|---|---|---|---|---|---|---|---|---|---|---|---|
| | | | | | | 2 | 1 | 6 | 4 | 5 | 3 |
| | | | | | | rpoB | rpoB | gyrA | fabG1 | fabG1 | rpoB |
| NoRS | NoSS | Prev | Pred | ME | VME | (4.47) | (3.62) | (1.85) | (0.56) | (0.56) | (0.52) |
| 5 | 357 | 63.73 | S | 0 | 5 | 1 | 1 | 1 | 1 | 1 | 1 |
| 42 | 1 | 7.57 | R | 1 | 0 | 0 | 1 | 0 | 0 | 0 | 0 |
| 22 | 19 | 7.22 | R | 19 | 0 | 0 | 1 | 1 | 1 | 1 | 0 |
| 0 | 32 | 5.63 | S | 0 | 0 | 1 | 1 | 1 | 0 | 0 | 1 |
| 22 | 0 | 3.87 | R | 0 | 0 | 0 | 1 | 0 | 1 | 1 | 0 |
| 12 | 3 | 2.64 | R | 3 | 0 | 0 | 1 | 1 | 0 | 0 | 0 |
| 1 | 13 | 2.46 | S | 0 | 1 | 1 | 0 | 1 | 1 | 1 | 0 |
| 9 | 0 | 1.58 | R | 0 | 0 | 1 | 0 | 0 | 0 | 0 | 1 |
| 7 | 1 | 1.41 | R | 1 | 0 | 1 | 0 | 0 | 1 | 1 | 1 |
| 1 | 3 | 0.7 | S | 0 | 1 | 1 | 0 | 1 | 1 | 1 | 1 |
| 1 | 3 | 0.7 | S | 0 | 1 | 1 | 1 | 1 | 1 | 1 | 0 |
| 1 | 2 | 0.53 | S | 0 | 1 | 1 | 1 | 0 | 1 | 1 | 1 |
| 2 | 0 | 0.35 | R | 0 | 0 | 1 | 0 | 1 | 0 | 0 | 1 |
| 2 | 0 | 0.35 | S | 0 | 2 | 1 | 1 | 1 | 0 | 0 | 0 |
| 1 | 0 | 0.18 | R | 0 | 0 | 0 | 0 | 1 | 1 | 1 | 0 |
| 0 | 1 | 0.18 | R | 1 | 0 | 0 | 1 | 1 | 1 | 1 | 1 |
| 1 | 0 | 0.18 | R | 0 | 0 | 1 | 0 | 0 | 0 | 0 | 0 |
| 1 | 0 | 0.18 | S | 0 | 1 | 1 | 0 | 1 | 0 | 0 | 0 |
| 1 | 0 | 0.18 | S | 0 | 1 | 1 | 1 | 0 | 0 | 0 | 0 |
| 1 | 0 | 0.18 | S | 0 | 1 | 1 | 1 | 0 | 0 | 0 | 1 |
| 1 | 0 | 0.18 | S | 0 | 1 | 1 | 1 | 0 | 1 | 1 | 0 |

### Interpretation of Results

In this model, the predominant haplotype is the one in which the presence of the sequences SEQ ID NOs: 1 to 6 is observed. This haplotype is observed in 362 genomes (357+5), i.e. 63.73% of the 568 validation genomes. Therefore, determining the presence of the sequences SEQ ID NOs: 1 to 6 leads to the correct determination (or prediction) of the “sensitivity” for 357 genomes, but with 5 VME.

### Performance of the Model

Overall, this model makes it possible to predict, on the basis of the presence or absence of the sequences SEQ ID NOs: 1 to 6, the sensitivity or resistance of a Mycobacterium tuberculosis strain with a specificity of 94.25%, a sensitivity of 88.72% and an error rate of 7.04% (ME and VME).

2.4 rpoB-fabG1-gyrA-Rv1042c/Rv1149 Model

Table 4 below presents the susceptibility results by the detection of the presence of mutations in the rpoB, fabG1, gyrA, Rv1042c and Rv1149 genes of the strain by demonstrating the presence or absence of the sequences SEQ ID NOs: 1 to 8.

TABLE 4

| | | | | | | SEQ ID NO: | | | | | | | |
|---|---|---|---|---|---|---|---|---|---|---|---|---|---|
| | | | | | | | | | 7 | | | 8 | |
| | | | | | | 2 | 1 | 6 | Rv1042c/ | 4 | 5 | Rv1042c/ | 3 |
| | | | | | | rpoB | rpoB | gyrA | Rv1149 | fabG1 | fabG1 | Rv1149 | rpoB |
| NoRS | NoSS | Prev | Pred | ME | VME | (3.99) | (3.07) | (1.8) | (1.24) | (0.4) | (0.4) | (0.61) | (0.48) |
| 1 | 186 | 32.92 | S | 0 | 1 | 1 | 1 | 1 | 1 | 1 | 1 | 0 | 1 |
| 1 | 85 | 15.14 | S | 0 | 1 | 1 | 1 | 1 | 0 | 1 | 1 | 0 | 1 |
| 3 | 82 | 14.96 | S | 0 | 3 | 1 | 1 | 1 | 1 | 1 | 1 | 1 | 1 |
| 37 | 1 | 6.69 | R | 1 | 0 | 0 | 1 | 0 | 1 | 0 | 0 | 1 | 0 |
| 15 | 7 | 3.87 | R | 7 | 0 | 0 | 1 | 1 | 1 | 1 | 1 | 1 | 0 |
| 0 | 19 | 3.35 | S | 0 | 0 | 1 | 1 | 1 | 1 | 0 | 0 | 0 | 1 |
| 18 | 0 | 3.17 | R | 0 | 0 | 0 | 1 | 0 | 1 | 1 | 1 | 1 | 0 |
| 6 | 9 | 2.64 | R | 9 | 0 | 0 | 1 | 1 | 1 | 1 | 1 | 0 | 0 |
| 11 | 0 | 1.94 | R | 0 | 0 | 0 | 1 | 1 | 1 | 0 | 0 | 1 | 0 |
| 9 | 0 | 1.58 | R | 0 | 0 | 1 | 0 | 0 | 1 | 0 | 0 | 1 | 1 |
| 0 | 7 | 1.23 | S | 0 | 0 | 1 | 0 | 1 | 1 | 1 | 1 | 0 | 0 |
| 0 | 7 | 1.23 | S | 0 | 0 | 1 | 1 | 1 | 1 | 0 | 0 | 1 | 1 |
| 6 | 0 | 1.06 | R | 0 | 0 | 1 | 0 | 0 | 1 | 1 | 1 | 1 | 1 |
| 1 | 5 | 1.06 | S | 0 | 1 | 1 | 0 | 1 | 1 | 1 | 1 | 1 | 0 |
| 0 | 6 | 1.06 | S | 0 | 0 | 1 | 1 | 1 | 0 | 0 | 0 | 0 | 1 |
| 5 | 0 | 0.88 | R | 0 | 0 | 0 | 1 | 0 | 1 | 0 | 0 | 0 | 0 |
| 1 | 3 | 0.7 | S | 0 | 1 | 0 | 1 | 1 | 0 | 1 | 1 | 0 | 0 |
| 1 | 3 | 0.7 | R | 3 | 0 | 0 | 1 | 1 | 1 | 0 | 0 | 0 | 0 |
| 0 | 4 | 0.7 | S | 0 | 0 | 1 | 1 | 1 | 0 | 1 | 1 | 1 | 1 |
| 3 | 0 | 0.53 | R | 0 | 0 | 0 | 1 | 0 | 1 | 1 | 1 | 0 | 0 |
| 1 | 2 | 0.53 | R | 2 | 0 | 1 | 0 | 1 | 1 | 1 | 1 | 1 | 1 |
| 1 | 2 | 0.53 | S | 0 | 1 | 1 | 1 | 1 | 1 | 1 | 1 | 0 | 0 |
| 1 | 1 | 0.35 | R | 1 | 0 | 1 | 0 | 0 | 1 | 1 | 1 | 0 | 1 |
| 2 | 0 | 0.35 | R | 0 | 0 | 1 | 0 | 1 | 1 | 0 | 0 | 1 | 1 |
| 1 | 1 | 0.35 | S | 0 | 1 | 1 | 1 | 0 | 1 | 1 | 1 | 0 | 1 |
| 1 | 0 | 0.18 | R | 0 | 0 | 0 | 0 | 1 | 1 | 1 | 1 | 1 | 0 |
| 1 | 0 | 0.18 | R | 0 | 0 | 0 | 1 | 0 | 0 | 1 | 1 | 0 | 0 |
| 0 | 1 | 0.18 | S | 0 | 0 | 0 | 1 | 1 | 0 | 1 | 1 | 0 | 1 |
| 1 | 0 | 0.18 | R | 0 | 0 | 1 | 0 | 0 | 1 | 0 | 0 | 0 | 0 |
| 0 | 1 | 0.18 | S | 0 | 0 | 1 | 0 | 1 | 0 | 1 | 1 | 0 | 0 |
| 1 | 0 | 0.18 | S | 0 | 1 | 1 | 0 | 1 | 1 | 0 | 0 | 0 | 0 |
| 0 | 1 | 0.18 | S | 0 | 0 | 1 | 0 | 1 | 1 | 1 | 1 | 0 | 1 |
| 1 | 0 | 0.18 | S | 0 | 1 | 1 | 1 | 0 | 1 | 0 | 0 | 0 | 0 |
| 1 | 0 | 0.18 | S | 0 | 1 | 1 | 1 | 0 | 1 | 0 | 0 | 1 | 1 |
| 1 | 0 | 0.18 | S | 0 | 1 | 1 | 1 | 0 | 1 | 1 | 1 | 1 | 0 |
| 0 | 1 | 0.18 | S | 0 | 0 | 1 | 1 | 0 | 1 | 1 | 1 | 1 | 1 |
| 1 | 0 | 0.18 | S | 0 | 1 | 1 | 1 | 1 | 1 | 0 | 0 | 0 | 0 |
| 1 | 0 | 0.18 | S | 0 | 1 | 1 | 1 | 1 | 1 | 0 | 0 | 1 | 0 |
| 0 | 1 | 0.18 | S | 0 | 0 | 1 | 1 | 1 | 1 | 1 | 1 | 1 | 0 |

### Interpretation of Results

In this model, the predominant haplotype is the one in which the presence of the sequences SEQ ID NOs: 1 to 6 and SEQ ID NO: 7, and the absence of the sequence SEQ ID NO: 8, is observed. This haplotype is observed in 187 genomes (186+1), i.e. 32.92% of the 568 validation genomes.

Therefore, determining the presence of the sequences SEQ ID NOs: 1 to 6 and SEQ ID NO: 7 and the absence of the sequence SEQ ID NO:8 leads to the correct determination (or prediction) of the “sensitivity” for 186 genomes, with only 1 VME.

Similarly, the haplotype in which the presence of the sequences SEQ ID NOs: 1 to 6 and the absence of the sequences SEQ ID NO: 7 and 8 is detected, is observed in 86 genomes, i.e. 14.14% of the validation genomes. The determination of the presence and absence of said sequences thus makes it possible to predict the “sensitivity” for 85 genomes, with only 1 VME.

### Performance of the Model

Overall, this model makes it possible to predict, on the basis of the presence or absence of the sequences SEQ ID NOs: 1 to 8, the sensitivity or resistance of a Mycobacterium tuberculosis strain with a specificity of 88.72%, a sensitivity of 94.71% and an error rate of 6.69% (ME and VME).

2.5 rpoB-fabG1-gyrA-Rv1042c/Rv1149-embB Model

Table 5 below presents the susceptibility results by the detection of the presence of mutations in the rpoB, fabG1, gyrA, Rv1042c, Rv1149 and embB genes of the strain by demonstrating the presence or absence of the sequences SEQ ID NOs: 1 to 9.

TABLE 5

| | | | | | | SEQ ID NO: | | | | | | | | |
|---|---|---|---|---|---|---|---|---|---|---|---|---|---|---|
| | | | | | | | | | 7 | | | 8 | | |
| | | | | | | 2 | 1 | 6 | Rv1042c/ | 4 | 5 | Rv1042c/ | 9 | 3 |
| | | | | | | rpoB | rpoB | gyrA | Rv1149 | fabG1 | fabG1 | Rv1149 | embB | rpoB |
| NoRS | NoSS | Prev | Pred | ME | VME | (3.2) | (2.57) | (1.63) | (1.19) | (0.44) | (0.44) | (0.74) | (1.27) | (0.35) |
| 1 | 185 | 32.75 | S | 0 | 1 | 1 | 1 | 1 | 1 | 1 | 1 | 0 | 1 | 1 |
| 1 | 84 | 14.96 | S | 0 | 1 | 1 | 1 | 1 | 0 | 1 | 1 | 0 | 1 | 1 |
| 3 | 82 | 14.96 | S | 0 | 3 | 1 | 1 | 1 | 1 | 1 | 1 | 1 | 1 | 1 |
| 31 | 0 | 5.46 | R | 0 | 0 | 0 | 1 | 0 | 1 | 0 | 0 | 1 | 0 | 0 |
| 0 | 19 | 3.35 | S | 0 | 0 | 1 | 1 | 1 | 1 | 0 | 0 | 0 | 1 | 1 |
| 10 | 3 | 2.29 | R | 3 | 0 | 0 | 1 | 1 | 1 | 1 | 1 | 1 | 1 | 0 |
| 4 | 7 | 1.94 | S | 0 | 4 | 0 | 1 | 1 | 1 | 1 | 1 | 0 | 1 | 0 |
| 10 | 0 | 1.76 | R | 0 | 0 | 0 | 1 | 0 | 1 | 1 | 1 | 1 | 0 | 0 |
| 10 | 0 | 1.76 | R | 0 | 0 | 0 | 1 | 1 | 1 | 0 | 0 | 1 | 0 | 0 |
| 5 | 4 | 1.58 | R | 4 | 0 | 0 | 1 | 1 | 1 | 1 | 1 | 1 | 0 | 0 |
| 9 | 0 | 1.58 | R | 0 | 0 | 1 | 0 | 0 | 1 | 0 | 0 | 1 | 0 | 1 |
| 8 | 0 | 1.41 | R | 0 | 0 | 0 | 1 | 0 | 1 | 1 | 1 | 1 | 1 | 0 |
| 6 | 1 | 1.23 | R | 1 | 0 | 0 | 1 | 0 | 1 | 0 | 0 | 1 | 1 | 0 |
| 0 | 7 | 1.23 | S | 0 | 0 | 1 | 0 | 1 | 1 | 1 | 1 | 0 | 1 | 0 |
| 0 | 6 | 1.06 | S | 0 | 0 | 1 | 1 | 1 | 0 | 0 | 0 | 0 | 1 | 1 |
| 0 | 6 | 1.06 | S | 0 | 0 | 1 | 1 | 1 | 1 | 0 | 0 | 1 | 1 | 1 |
| 5 | 0 | 0.88 | R | 0 | 0 | 1 | 0 | 0 | 1 | 1 | 1 | 1 | 1 | 1 |
| 4 | 0 | 0.7 | R | 0 | 0 | 0 | 1 | 0 | 1 | 0 | 0 | 0 | 1 | 0 |
| 2 | 2 | 0.7 | R | 2 | 0 | 0 | 1 | 1 | 1 | 1 | 1 | 0 | 0 | 0 |
| 0 | 4 | 0.7 | S | 0 | 0 | 1 | 1 | 1 | 0 | 1 | 1 | 1 | 1 | 1 |
| 3 | 0 | 0.53 | R | 0 | 0 | 0 | 1 | 0 | 1 | 1 | 1 | 0 | 0 | 0 |
| 0 | 3 | 0.53 | S | 0 | 0 | 0 | 1 | 1 | 0 | 1 | 1 | 0 | 1 | 0 |
| 0 | 3 | 0.53 | S | 0 | 0 | 0 | 1 | 1 | 1 | 0 | 0 | 0 | 1 | 0 |
| 0 | 3 | 0.53 | R | 3 | 0 | 1 | 0 | 1 | 1 | 1 | 1 | 1 | 0 | 0 |
| 1 | 2 | 0.53 | S | 0 | 1 | 1 | 0 | 1 | 1 | 1 | 1 | 1 | 1 | 0 |
| 1 | 1 | 0.35 | R | 1 | 0 | 1 | 0 | 0 | 1 | 1 | 1 | 0 | 0 | 1 |
| 1 | 1 | 0.35 | R | 1 | 0 | 1 | 0 | 1 | 1 | 1 | 1 | 1 | 0 | 1 |
| 1 | 1 | 0.35 | S | 0 | 1 | 1 | 1 | 1 | 1 | 1 | 1 | 0 | 0 | 0 |
| 1 | 0 | 0.18 | R | 0 | 0 | 0 | 0 | 1 | 1 | 1 | 1 | 1 | 1 | 0 |
| 1 | 0 | 0.18 | R | 0 | 0 | 0 | 1 | 0 | 0 | 1 | 1 | 0 | 0 | 0 |
| 1 | 0 | 0.18 | R | 0 | 0 | 0 | 1 | 0 | 1 | 0 | 0 | 0 | 0 | 0 |
| 1 | 0 | 0.18 | S | 0 | 1 | 0 | 1 | 1 | 0 | 1 | 1 | 0 | 0 | 0 |
| 0 | 1 | 0.18 | S | 0 | 0 | 0 | 1 | 1 | 0 | 1 | 1 | 0 | 1 | 1 |
| 1 | 0 | 0.18 | R | 0 | 0 | 0 | 1 | 1 | 1 | 0 | 0 | 0 | 0 | 0 |
| 1 | 0 | 0.18 | R | 0 | 0 | 0 | 1 | 1 | 1 | 0 | 0 | 1 | 1 | 0 |
| 1 | 0 | 0.18 | R | 0 | 0 | 1 | 0 | 0 | 1 | 0 | 0 | 0 | 1 | 0 |
| 1 | 0 | 0.18 | R | 0 | 0 | 1 | 0 | 0 | 1 | 1 | 1 | 1 | 0 | 1 |
| 0 | 1 | 0.18 | S | 0 | 0 | 1 | 0 | 1 | 0 | 1 | 1 | 0 | 1 | 0 |
| 1 | 0 | 0.18 | R | 0 | 0 | 1 | 0 | 1 | 1 | 0 | 0 | 0 | 0 | 0 |
| 1 | 0 | 0.18 | R | 0 | 0 | 1 | 0 | 1 | 1 | 0 | 0 | 1 | 0 | 1 |
| 1 | 0 | 0.18 | R | 0 | 0 | 1 | 0 | 1 | 1 | 0 | 0 | 1 | 1 | 1 |
| 0 | 1 | 0.18 | S | 0 | 0 | 1 | 0 | 1 | 1 | 1 | 1 | 0 | 1 | 1 |
| 0 | 1 | 0.18 | S | 0 | 0 | 1 | 0 | 1 | 1 | 1 | 1 | 1 | 1 | 1 |
| 1 | 0 | 0.18 | S | 0 | 1 | 1 | 1 | 0 | 1 | 0 | 0 | 0 | 1 | 0 |
| 1 | 0 | 0.18 | R | 0 | 0 | 1 | 1 | 0 | 1 | 0 | 0 | 1 | 0 | 1 |
| 1 | 0 | 0.18 | S | 0 | 1 | 1 | 1 | 0 | 1 | 1 | 1 | 0 | 0 | 1 |
| 0 | 1 | 0.18 | S | 0 | 0 | 1 | 1 | 0 | 1 | 1 | 1 | 0 | 1 | 1 |
| 1 | 0 | 0.18 | S | 0 | 1 | 1 | 1 | 0 | 1 | 1 | 1 | 1 | 0 | 0 |
| 0 | 1 | 0.18 | S | 0 | 0 | 1 | 1 | 0 | 1 | 1 | 1 | 1 | 1 | 1 |
| 0 | 1 | 0.18 | S | 0 | 0 | 1 | 1 | 1 | 0 | 1 | 1 | 0 | 0 | 1 |
| 1 | 0 | 0.18 | S | 0 | 1 | 1 | 1 | 1 | 1 | 0 | 0 | 0 | 0 | 0 |
| 1 | 0 | 0.18 | S | 0 | 1 | 1 | 1 | 1 | 1 | 0 | 0 | 1 | 0 | 0 |
| 0 | 1 | 0.18 | S | 0 | 0 | 1 | 1 | 1 | 1 | 0 | 0 | 1 | 0 | 1 |
| 0 | 1 | 0.18 | S | 0 | 0 | 1 | 1 | 1 | 1 | 1 | 1 | 0 | 0 | 1 |
| 0 | 1 | 0.18 | S | 0 | 0 | 1 | 1 | 1 | 1 | 1 | 1 | 0 | 1 | 0 |
| 0 | 1 | 0.18 | S | 0 | 0 | 1 | 1 | 1 | 1 | 1 | 1 | 1 | 1 | 0 |

### Interpretation of Results

In this model, the predominant haplotype is the one in which the presence of the sequences SEQ ID NOs: 1 to 6, 9 and SEQ ID NO: 7, and the absence of the sequence SEQ ID NO: 8, is observed. This haplotype is observed in 186 genomes (185+1), i.e. 32.75% of the 568 validation genomes.

Consequently, the determination of the presence and absence of said sequences in the genome of a Mycobacterium tuberculosis strain leads to the correct determination (or prediction) of the “sensitivity” for 185 genomes, with only 1 VME.

Similarly, the haplotype in which the presence of all of the sequences SEQ ID NOs: 1 to 9 is detected is observed in 85 genomes, i.e. 14.96% of the validation genomes. The determination of the presence and absence of said sequences thus makes it possible to predict the “sensitivity” for 82 genomes, with 3 VME.

### Performance of the Model

Overall, this model makes it possible to predict, on the basis of the presence or absence of the sequences SEQ ID NO: 1 to 9, the sensitivity or resistance of a Mycobacterium tuberculosis strain with a specificity of 87.22%, a sensitivity of 96.55% and an error rate of 5.63% (ME and VME).

2.6 poB-fabG1-gyrA-Rv1042c/Rv1149-embB-pncA-katG Model

Table 6 below presents the susceptibility results by the detection of the presence of mutations in the rpoB, fabG1, gyrA, Rv1042c, Rv1149, embB, pncA and katG genes of the strain by demonstrating the presence or absence of the sequences SEQ ID NOs: 1 to 12.

TABLE 6

| | | | | | | SEQ ID NO: | | | | | | | | | | | |
|---|---|---|---|---|---|---|---|---|---|---|---|---|---|---|---|---|---|
| | | | | | | | | | | | | 7 | | | 8 | | |
| | | | | | | 10 | 2 | 11 | 1 | 6 | 12 | Rv1042c/ | 4 | 5 | Rv1042c/ | 9 | 3 |
| | | | | | | pncA | rpoB | pncA | rpoB | gyrA | katG | Rv1149 | fabG1 | fabG1 | Rv1149 | embB | rpoB |
| NoRS | NoSS | Prev | Pred | ME | VME | (3.64) | (2.52) | (2.61) | (2.04) | (1.46) | (1.23) | (1.06) | (0.91) | (0.91) | (0.84) | (0.71) | (0.43) |
| 1 | 163 | 28.87 | S | 0 | 1 | 1 | 1 | 1 | 1 | 1 | 1 | 1 | 1 | 1 | 0 | 1 | 1 |
| 1 | 75 | 13.38 | S | 0 | 1 | 1 | 1 | 1 | 1 | 1 | 1 | 0 | 1 | 1 | 0 | 1 | 1 |
| 3 | 72 | 13.2 | S | 0 | 3 | 1 | 1 | 1 | 1 | 1 | 1 | 1 | 1 | 1 | 1 | 1 | 1 |
| 0 | 22 | 3.87 | S | 0 | 0 | 1 | 1 | 1 | 1 | 1 | 0 | 1 | 1 | 1 | 0 | 1 | 1 |
| 0 | 19 | 3.35 | S | 0 | 0 | 1 | 1 | 1 | 1 | 1 | 1 | 1 | 0 | 0 | 0 | 1 | 1 |
| 14 | 0 | 2.46 | R | 0 | 0 | 1 | 0 | 0 | 1 | 0 | 0 | 1 | 0 | 0 | 1 | 0 | 0 |
| 11 | 0 | 1.94 | R | 0 | 0 | 1 | 0 | 1 | 1 | 0 | 1 | 1 | 0 | 0 | 1 | 0 | 0 |
| 9 | 0 | 1.58 | R | 0 | 0 | 1 | 1 | 1 | 0 | 0 | 0 | 1 | 0 | 0 | 1 | 0 | 1 |
| 0 | 9 | 1.58 | S | 0 | 0 | 1 | 1 | 1 | 1 | 1 | 0 | 0 | 1 | 1 | 0 | 1 | 1 |
| 0 | 9 | 1.58 | S | 0 | 0 | 1 | 1 | 1 | 1 | 1 | 0 | 1 | 1 | 1 | 1 | 1 | 1 |
| 8 | 0 | 1.41 | R | 0 | 0 | 1 | 0 | 1 | 1 | 0 | 0 | 1 | 1 | 1 | 1 | 0 | 0 |
| 5 | 3 | 1.41 | R | 3 | 0 | 1 | 0 | 1 | 1 | 1 | 0 | 1 | 1 | 1 | 1 | 0 | 0 |
| 7 | 1 | 1.41 | R | 1 | 0 | 1 | 0 | 1 | 1 | 1 | 0 | 1 | 1 | 1 | 1 | 1 | 0 |
| 0 | 7 | 1.23 | S | 0 | 0 | 1 | 1 | 1 | 0 | 1 | 1 | 1 | 1 | 1 | 0 | 1 | 0 |
| 6 | 0 | 1.06 | R | 0 | 0 | 1 | 0 | 1 | 1 | 0 | 0 | 1 | 1 | 1 | 1 | 1 | 0 |
| 5 | 1 | 1.06 | R | 1 | 0 | 1 | 0 | 1 | 1 | 0 | 1 | 1 | 0 | 0 | 1 | 1 | 0 |
| 0 | 6 | 1.06 | S | 0 | 0 | 1 | 1 | 1 | 1 | 1 | 1 | 1 | 0 | 0 | 1 | 1 | 1 |
| 1 | 4 | 0.88 | S | 0 | 1 | 1 | 0 | 1 | 1 | 1 | 0 | 1 | 1 | 1 | 0 | 1 | 0 |
| 0 | 5 | 0.88 | S | 0 | 0 | 1 | 1 | 1 | 1 | 1 | 1 | 0 | 0 | 0 | 0 | 1 | 1 |
| 4 | 0 | 0.7 | R | 0 | 0 | 1 | 0 | 1 | 1 | 0 | 1 | 1 | 0 | 0 | 0 | 1 | 0 |
| 4 | 0 | 0.7 | R | 0 | 0 | 1 | 1 | 1 | 0 | 0 | 0 | 1 | 1 | 1 | 1 | 1 | 1 |
| 3 | 0 | 0.53 | R | 0 | 0 | 1 | 0 | 0 | 1 | 0 | 1 | 1 | 0 | 0 | 1 | 0 | 0 |
| 3 | 0 | 0.53 | R | 0 | 0 | 1 | 0 | 0 | 1 | 1 | 0 | 1 | 0 | 0 | 1 | 0 | 0 |
| 2 | 1 | 0.53 | R | 1 | 0 | 1 | 0 | 0 | 1 | 1 | 0 | 1 | 1 | 1 | 0 | 1 | 0 |
| 3 | 0 | 0.53 | R | 0 | 0 | 1 | 0 | 1 | 1 | 0 | 0 | 1 | 0 | 0 | 1 | 0 | 0 |
| 3 | 0 | 0.53 | R | 0 | 0 | 1 | 0 | 1 | 1 | 1 | 0 | 1 | 0 | 0 | 1 | 0 | 0 |
| 2 | 1 | 0.53 | R | 1 | 0 | 1 | 0 | 1 | 1 | 1 | 0 | 1 | 1 | 1 | 0 | 0 | 0 |
| 1 | 2 | 0.53 | S | 0 | 1 | 1 | 0 | 1 | 1 | 1 | 1 | 1 | 1 | 1 | 0 | 1 | 0 |
| 0 | 3 | 0.53 | R | 3 | 0 | 1 | 1 | 1 | 0 | 1 | 0 | 1 | 1 | 1 | 1 | 0 | 0 |
| 1 | 2 | 0.53 | S | 0 | 1 | 1 | 1 | 1 | 0 | 1 | 1 | 1 | 1 | 1 | 1 | 1 | 0 |
| 0 | 3 | 0.53 | S | 0 | 0 | 1 | 1 | 1 | 1 | 1 | 1 | 0 | 1 | 1 | 1 | 1 | 1 |
| 2 | 0 | 0.35 | R | 0 | 0 | 1 | 0 | 0 | 1 | 0 | 0 | 1 | 1 | 1 | 1 | 0 | 0 |
| 2 | 0 | 0.35 | R | 0 | 0 | 1 | 0 | 0 | 1 | 0 | 0 | 1 | 1 | 1 | 1 | 1 | 0 |
| 2 | 0 | 0.35 | R | 0 | 0 | 1 | 0 | 0 | 1 | 1 | 0 | 1 | 1 | 1 | 1 | 1 | 0 |
| 2 | 0 | 0.35 | R | 0 | 0 | 1 | 0 | 1 | 1 | 0 | 0 | 1 | 1 | 1 | 0 | 0 | 0 |
| 0 | 2 | 0.35 | S | 0 | 0 | 1 | 0 | 1 | 1 | 1 | 1 | 0 | 1 | 1 | 0 | 1 | 0 |
| 0 | 2 | 0.35 | S | 0 | 0 | 1 | 0 | 1 | 1 | 1 | 1 | 1 | 0 | 0 | 0 | 1 | 0 |
| 2 | 0 | 0.35 | R | 0 | 0 | 1 | 0 | 1 | 1 | 1 | 1 | 1 | 0 | 0 | 1 | 0 | 0 |
| 0 | 2 | 0.35 | S | 0 | 0 | 1 | 0 | 1 | 1 | 1 | 1 | 1 | 1 | 1 | 1 | 1 | 0 |
| 1 | 1 | 0.35 | S | 0 | 1 | 1 | 1 | 1 | 0 | 1 | 1 | 1 | 1 | 1 | 1 | 0 | 1 |
| 1 | 1 | 0.35 | S | 0 | 1 | 1 | 1 | 1 | 1 | 1 | 0 | 1 | 1 | 1 | 0 | 0 | 0 |
| 1 | 0 | 0.18 | R | 0 | 0 | 0 | 0 | 1 | 1 | 0 | 1 | 1 | 0 | 0 | 0 | 0 | 0 |
| 1 | 0 | 0.18 | R | 0 | 0 | 0 | 0 | 1 | 1 | 1 | 1 | 1 | 0 | 0 | 0 | 0 | 0 |
| 1 | 0 | 0.18 | R | 0 | 0 | 0 | 0 | 1 | 1 | 1 | 1 | 1 | 0 | 0 | 1 | 0 | 0 |
| 1 | 0 | 0.18 | R | 0 | 0 | 0 | 0 | 1 | 1 | 1 | 1 | 1 | 1 | 1 | 1 | 1 | 0 |
| 1 | 0 | 0.18 | R | 0 | 0 | 0 | 1 | 0 | 1 | 0 | 1 | 1 | 1 | 1 | 0 | 0 | 1 |
| 1 | 0 | 0.18 | R | 0 | 0 | 1 | 0 | 0 | 1 | 0 | 1 | 1 | 0 | 0 | 1 | 1 | 0 |
| 1 | 0 | 0.18 | R | 0 | 0 | 1 | 0 | 0 | 1 | 1 | 1 | 1 | 0 | 0 | 1 | 0 | 0 |
| 1 | 0 | 0.18 | R | 0 | 0 | 1 | 0 | 1 | 0 | 1 | 1 | 1 | 1 | 1 | 1 | 1 | 0 |
| 1 | 0 | 0.18 | R | 0 | 0 | 1 | 0 | 1 | 1 | 0 | 0 | 0 | 1 | 1 | 0 | 0 | 0 |
| 1 | 0 | 0.18 | R | 0 | 0 | 1 | 0 | 1 | 1 | 0 | 1 | 1 | 1 | 1 | 0 | 0 | 0 |
| 0 | 1 | 0.18 | S | 0 | 0 | 1 | 0 | 1 | 1 | 1 | 0 | 0 | 1 | 1 | 0 | 1 | 0 |
| 0 | 1 | 0.18 | R | 1 | 0 | 1 | 0 | 1 | 1 | 1 | 0 | 1 | 0 | 0 | 0 | 1 | 0 |
| 1 | 0 | 0.18 | S | 0 | 1 | 1 | 0 | 1 | 1 | 1 | 1 | 0 | 1 | 1 | 0 | 0 | 0 |
| 0 | 1 | 0.18 | S | 0 | 0 | 1 | 0 | 1 | 1 | 1 | 1 | 0 | 1 | 1 | 0 | 1 | 1 |
| 1 | 0 | 0.18 | S | 0 | 1 | 1 | 0 | 1 | 1 | 1 | 1 | 1 | 0 | 0 | 1 | 1 | 0 |
| 0 | 1 | 0.18 | S | 0 | 0 | 1 | 0 | 1 | 1 | 1 | 1 | 1 | 1 | 1 | 0 | 0 | 0 |
| 0 | 1 | 0.18 | S | 0 | 0 | 1 | 0 | 1 | 1 | 1 | 1 | 1 | 1 | 1 | 1 | 0 | 0 |
| 1 | 0 | 0.18 | R | 0 | 0 | 1 | 1 | 0 | 0 | 0 | 0 | 1 | 1 | 1 | 0 | 0 | 1 |
| 0 | 1 | 0.18 | S | 0 | 0 | 1 | 1 | 0 | 1 | 1 | 1 | 1 | 1 | 1 | 1 | 1 | 1 |
| 1 | 0 | 0.18 | R | 0 | 0 | 1 | 1 | 1 | 0 | 0 | 0 | 1 | 0 | 0 | 0 | 1 | 0 |
| 0 | 1 | 0.18 | R | 1 | 0 | 1 | 1 | 1 | 0 | 0 | 0 | 1 | 1 | 1 | 0 | 0 | 1 |
| 1 | 0 | 0.18 | R | 0 | 0 | 1 | 1 | 1 | 0 | 0 | 0 | 1 | 1 | 1 | 1 | 0 | 1 |
| 1 | 0 | 0.18 | R | 0 | 0 | 1 | 1 | 1 | 0 | 0 | 1 | 1 | 1 | 1 | 1 | 1 | 1 |
| 0 | 1 | 0.18 | S | 0 | 0 | 1 | 1 | 1 | 0 | 1 | 0 | 0 | 1 | 1 | 0 | 1 | 0 |
| 1 | 0 | 0.18 | R | 0 | 0 | 1 | 1 | 1 | 0 | 1 | 0 | 1 | 0 | 0 | 0 | 0 | 0 |
| 1 | 0 | 0.18 | R | 0 | 0 | 1 | 1 | 1 | 0 | 1 | 0 | 1 | 0 | 0 | 1 | 0 | 1 |
| 0 | 1 | 0.18 | R | 1 | 0 | 1 | 1 | 1 | 0 | 1 | 0 | 1 | 1 | 1 | 1 | 1 | 1 |
| 1 | 0 | 0.18 | S | 0 | 1 | 1 | 1 | 1 | 0 | 1 | 1 | 1 | 0 | 0 | 1 | 1 | 1 |
| 0 | 1 | 0.18 | S | 0 | 0 | 1 | 1 | 1 | 0 | 1 | 1 | 1 | 1 | 1 | 0 | 1 | 1 |
| 1 | 0 | 0.18 | S | 0 | 1 | 1 | 1 | 1 | 1 | 0 | 0 | 1 | 0 | 0 | 0 | 1 | 0 |
| 1 | 0 | 0.18 | R | 0 | 0 | 1 | 1 | 1 | 1 | 0 | 1 | 1 | 0 | 0 | 1 | 0 | 1 |
| 0 | 1 | 0.18 | S | 0 | 0 | 1 | 1 | 1 | 1 | 0 | 1 | 1 | 1 | 1 | 0 | 1 | 1 |
| 1 | 0 | 0.18 | S | 0 | 1 | 1 | 1 | 1 | 1 | 0 | 1 | 1 | 1 | 1 | 1 | 0 | 0 |
| 0 | 1 | 0.18 | S | 0 | 0 | 1 | 1 | 1 | 1 | 0 | 1 | 1 | 1 | 1 | 1 | 1 | 1 |
| 0 | 1 | 0.18 | S | 0 | 0 | 1 | 1 | 1 | 1 | 1 | 0 | 0 | 0 | 0 | 0 | 1 | 1 |
| 0 | 1 | 0.18 | S | 0 | 0 | 1 | 1 | 1 | 1 | 1 | 0 | 0 | 1 | 1 | 1 | 1 | 1 |
| 1 | 0 | 0.18 | S | 0 | 1 | 1 | 1 | 1 | 1 | 1 | 0 | 1 | 0 | 0 | 0 | 0 | 0 |
| 0 | 1 | 0.18 | S | 0 | 0 | 1 | 1 | 1 | 1 | 1 | 0 | 1 | 1 | 1 | 0 | 0 | 1 |
| 0 | 1 | 0.18 | S | 0 | 0 | 1 | 1 | 1 | 1 | 1 | 0 | 1 | 1 | 1 | 0 | 1 | 0 |
| 0 | 1 | 0.18 | S | 0 | 0 | 1 | 1 | 1 | 1 | 1 | 0 | 1 | 1 | 1 | 1 | 1 | 0 |
| 0 | 1 | 0.18 | S | 0 | 0 | 1 | 1 | 1 | 1 | 1 | 1 | 0 | 1 | 1 | 0 | 0 | 1 |
| 1 | 0 | 0.18 | S | 0 | 1 | 1 | 1 | 1 | 1 | 1 | 1 | 1 | 0 | 0 | 1 | 0 | 0 |
| 0 | 1 | 0.18 | S | 0 | 0 | 1 | 1 | 1 | 1 | 1 | 1 | 1 | 0 | 0 | 1 | 0 | 1 |

### Interpretation of Results

In this model, the predominant haplotype is the one in which the presence of the sequences SEQ ID NOs: 1 to 6, 9 to 12 and SEQ ID NO: 7, and the absence of the sequence SEQ ID NO: 8, is observed. This haplotype is observed in 164 genomes (163+1), i.e. 28.87% of the 568 validation genomes.

Consequently, the determination of the presence and absence of said sequences in the genome of a Mycobacterium tuberculosis strain leads to the correct determination (or prediction) of the “sensitivity” for 163 genomes, with only 1 VME.

Similarly, the haplotype in which the presence of all of the sequences SEQ ID NOs: 1 to 12 is detected is observed in 75 genomes, i.e. 13.2% of the validation genomes. The determination of the presence and absence of said sequences thus makes it possible to predict the “sensitivity” for 72 genomes, with 3 VME.

### Performance of the Model

Overall, this model makes it possible to predict, on the basis of the presence or absence of the sequences SEQ ID NOs: 1 to 12, the sensitivity or resistance of a Mycobacterium tuberculosis strain with a specificity of 88.72%, a sensitivity of 97.01% and an error rate of 5.28% (ME and VME).

### Example 2: Description of the Sequences Useful for Implementing the Method According to the Invention

| | | SEQ ID |
|---|---|---|
| Name | Nucleic acid sequence | NO: |
| rpoB unitig 1 | GACCAGAACAACCCGCTGTCGGGGTTGACCC | 1 |
| rpoB unitig 2 | GACAGACCGCCGGGCCCCAGCGCCGACAGTCG | 2 |
| rpoB unitig 3 | GACCCACAAGCGCCGACTGTCGGCGCTGGGGCCCG | 3 |
| FabG1 unitig 1 | CCGACAACCTATCGTCTCGCCGCGGCCGGGCC | 4 |
| FabG1 unitig 2 | GGCAGTCACCCCGACAACCTATCGTCTCGCCGCGG | 5 |
| | CCGGG | |
| gyrA unitig | ACCACCCGCACGGCGACGCGTCGATCTACGA | 6 |
| Rv1042v/Rv11 | CGGCATCAACGGCTCGACCACGGCCCAGAACTCGT | 7 |
| 49 unitig 1 | CCGAAATCACACCC | |
| Rv1042v/Rv11 | GCGTCACCGGCCAATCCTCGCTGGCCAGTACC | 8 |
| 49 unitig 2 | | |
| embB unitig | CGGCGACTCGGGCCATGCCCAGGATGTAGCCGTCG | 9 |
| | TCCGACG | |
| pncA unitig 1 | AACGCGGCGTCGATGAGGTCGATGTGGTCGGTA | 10 |
| pncA unitig 2 | CAGAAGTCGTTCTGCACGTCGACGATGATCA | 11 |
| katG unitig | GCACCGGAACCGGTAAGGACGCGATCACCAGCGGC | 12 |
| | ATCGAGG | |

1. A method for determining the susceptibility of a strain of the species Mycobacterium tuberculosis to pyrazinamide, the method comprising the following steps:
determining the presence, in the genome of said strain, of at least one mutation in the rpoB gene between positions 761112 and 761182,
determining the sensitivity or resistance of said strain on the basis of the presence or absence of the mutation.

2. The method as claimed in claim 1, wherein it also comprises determining the presence, in the genome of said strain, of at least one mutation in the fabG1 gene between positions 1673413 and 1673454.

3. The method as claimed in claim 1, wherein it also comprises determining the presence, in the genome of said strain, of at least one mutation in the gyrA gene between positions 7552 and 7582.

4. The method as claimed in claim 1, wherein it also comprises determining the presence, in the genome of said strain, of at least one mutation in the Rv1042c gene and the promoter region thereof, between positions 1165444 and 1165528, and/or at least one mutation in the Rv1149 gene and the promoter region thereof, between positions 1277873 and 1277957.

5. The method as claimed in claim 4, wherein it also comprises a step of determining the presence of at least one mutation in the Rv1042c gene between positions 1165444 and 1165492 and at least one mutation in the Rv1149 gene between positions 1277909 and 1277957, and/or a step of determining the presence of at least one mutation in the Rv1042c gene and the promoter region thereof, between positions 1165497 and 1165528, and at least one mutation in the Rv1149 gene and the promoter region thereof, between positions 1277873 and 1277904.

6. The method as claimed in claim 1, wherein it also comprises the following steps of:
determining the presence, in the genome of the strain, of at least one mutation in the embB gene between positions 4247581 and 4247622, and/or
determining the presence, in the genome of the strain, of at least one mutation in the pncA gene between positions 2288853 and 2289239, and/or
determining the presence, in the genome of the strain, of at least one mutation in the katG gene between positions 2155164 and 2155205.

7. The method as claimed in one of claim 1, wherein determining the presence of at least one mutation in the rpoB gene comprises determining the presence of at least one sequence selected from the sequences SEQ ID NO: 1, SEQ ID NO: 2 and SEQ ID NO: 3.

8. The method as claimed in claim 2, wherein determining the presence of at least one mutation in the fabG1 gene comprises determining the presence of at least one sequence selected from the sequences SEQ ID NO: 4 and SEQ ID NO: 5.

9. The method as claimed in claim 3, wherein determining the presence of at least one mutation in the gyrA gene comprises determining the presence of the sequence SEQ ID NO: 6.

10. The method as claimed in claim 4, wherein determining the presence of at least one mutation in the genomic region between positions 1165444 and 1165528 and/or of at least one mutation in the genomic region between positions 1277873 and 1277957 comprises determining the presence of at least one sequence selected from the sequences SEQ ID NO: 7 and SEQ ID NO: 8.

11. The method as claimed in claim 6, wherein determining the presence of at least one mutation in the embB gene comprises determining the presence of the sequence SEQ ID NO: 9.

12. The method as claimed in claim 6, wherein
determining the presence of at least one mutation in the pncA gene consists in determining the presence of at least one of the sequences SEQ ID NO: 10 and SEQ ID NO: 11,
determining the presence of at least one mutation in the katG gene consists in determining the presence of the sequence SEQ ID NO: 12.

13. The method as claimed in claim 12, wherein it comprises a step of determining the presence of the sequences SEQ ID NOs: 1 to 12 in the respective genes, and determining the sensitivity or resistance of said strain on the basis of the presence or absence of the sequences.

14. The method as claimed in claim 1, wherein the steps of determining the presence of mutations are carried out with respect to the wild-type reference genome of Mycobacterium tuberculosis H37Rv (reference NC_000962.3).

15. The method as claimed in claim 1, wherein the step of determining the presence of at least one mutation in the genes is carried out by a hybridization, amplification or sequencing method.

16. The method as claimed in claim 1, wherein it is implemented by a computer.

17. A data processing apparatus, comprising:
means for implementing the method as claimed in claim 16, means for determining the presence of mutations in the rpoB gene of the Mycobacterium tuberculosis strain between positions 761112 and 761182, and/or means making it possible to compare the sequence of said gene with the reference genome Mycobacterium tuberculosis H37Rv (reference NC_000962.3), and/or means for identifying the presence of the sequences SEQ ID NOs: 1 to 3 in the genome of the Mycobacterium tuberculosis strain, and/or means for providing output data regarding the presence or absence of the sequences when said means are implemented or controlled by a computer, or
a processor suitable for, or configured for, executing the computer-implemented method as claimed in claim 16.

18. A kit comprising means for detecting at least one sequence selected from the sequences SEQ ID NO: 1 to 3, and optionally at least one other sequence selected from the sequences SEQ ID NOs: 4 to 12, the means being primers and/or probes.

19. A method for determining the susceptibility of a Mycobacterium tuberculosis strain to pyrazinamide comprising assaying a sample using the kit as claimed in claim 18 to detect at least one sequence selected from the sequences SEQ ID NOS: 1 to 3.

20. A method for determining the susceptibility of a Mycobacterium tuberculosis strain to pyrazinamide comprising detecting the presence of at least one sequence selected from the sequences SEQ ID NOs 1 to 3, and optionally also detecting the presence of at least one of the sequences SEQ ID Nos 4 to 12.


SEQUENCE LISTING (12 sequences)

SEQ ID NO: 1 - DNA, 31 bp, Mycobacterium tuberculosis
  source 1..31: mol_type = genomic DNA
gaccagaacaacccgctgtcggggttgaccc

SEQ ID NO: 2 - DNA, 32 bp, Mycobacterium tuberculosis
  source 1..32: mol_type = genomic DNA
gacagaccgccgggccccagcgccgacagtcg

SEQ ID NO: 3 - DNA, 35 bp, Mycobacterium tuberculosis
  source 1..35: mol_type = genomic DNA
gacccacaagcgccgactgtcggcgctggggcccg

SEQ ID NO: 4 - DNA, 32 bp, Mycobacterium tuberculosis
  source 1..32: mol_type = genomic DNA
ccgacaacctatcgtctcgccgcggccgggcc

SEQ ID NO: 5 - DNA, 40 bp, Mycobacterium tuberculosis
  source 1..40: mol_type = genomic DNA
ggcagtcaccccgacaacctatcgtctcgccgcggccggg

SEQ ID NO: 6 - DNA, 31 bp, Mycobacterium tuberculosis
  source 1..31: mol_type = genomic DNA
accacccgcacggcgacgcgtcgatctacga

SEQ ID NO: 7 - DNA, 49 bp, Mycobacterium tuberculosis
  source 1..49: mol_type = genomic DNA
cggcatcaacggctcgaccacggcccagaactcgtccgaaatcacaccc

SEQ ID NO: 8 - DNA, 32 bp, Mycobacterium tuberculosis
  source 1..32: mol_type = genomic DNA
gcgtcaccggccaatcctcgctggccagtacc

SEQ ID NO: 9 - DNA, 42 bp, Mycobacterium tuberculosis
  source 1..42: mol_type = genomic DNA
cggcgactcgggccatgcccaggatgtagccgtcgtccgacg

SEQ ID NO: 10 - DNA, 33 bp, Mycobacterium tuberculosis
  source 1..33: mol_type = genomic DNA
aacgcggcgtcgatgaggtcgatgtggtcggta

SEQ ID NO: 11 - DNA, 31 bp, Mycobacterium tuberculosis
  source 1..31: mol_type = genomic DNA
cagaagtcgttctgcacgtcgacgatgatca

SEQ ID NO: 12 - DNA, 42 bp, Mycobacterium tuberculosis
  source 1..42: mol_type = genomic DNA
gcaccggaaccggtaaggacgcgatcaccagcggcatcgagg